(19) United States
(12) Patent Application Publication
(10) Pub. No.: US 2005/0180945 A1
(43) Pub. Date: Aug. 18, 2005

(54) Multivalent polymers with chan-terminating binding groups

(76) Inventors: Elliot L Chaikof, Atlanta, GA (US); Xue-Long Sun, Atlanta, GA (US)

Correspondence Address: GREENLEE WINNER AND SULLIVAN P C, 4875 PEARL EAST CIRCLE, SUITE 200, BOULDER, CO, 80301, US

(21) Appl. No.: 10/514,064

(22) PCT Filed: May 21, 2003
(86) PCT No.: PCT/US03/15901

Related U.S. Application Data
(60) Provisional application No. 60/382,512, filed on May 21, 2002.

(51) Int. Cl. A61K 31/785

(52) U.S. Cl. 424/78.27; 525/54.2

(57) ABSTRACT

Polymeric molecules including glycopolymers useful for biomolecular recognition processes are provided comprising anchoring groups by which they can be immobilized onto surfaces. These molecules are easily synthesized. They are broadly described as molecules comprising a polymer backbone with pendent multivalent groups attached to the polymer backbone and an anchoring group attached to the polymer backbone for covalently or noncovalently attaching the molecule to a surface or another molecule. Such polymeric molecules can be attached to other molecules or surfaces to provide a wide range of bioactive materials. In addition, this invention provides sulfated glycopolymers which are useful for reducing blood coagulation, stimulating growth factors to bind to their receptors, stimulating cell proliferation and preventing degradation of soluble growth factors under conditions of low pH, heat, and proteolytic enzymes.

### CROSS-REFERENCE TO RELATED APPLICATIONS

This application claims priority to U.S. application Ser. No. 60/382,512 filed May 21, 2002, which is incorporated herein by reference to the extent not inconsistent herewith.

### BACKGROUND

Cell surface proteoglycans and glycolipids collectively form a membrane-bound carbohydrate coating, often referred to as a glycocalyx. Membrane-associated polysaccharides are critical mediators of molecular recognition events via the interactions of unique oligosaccharide sequences with specific protein epitopes that maybe found on bacteria, viruses, and other cells, as well as on a variety of soluble and matrix-bound factors. (Wang, D. et al., Nat. Technol. 2002, 20, 275-281; Houseman, B. T. and Mrksich, M., Chem. Biol. 2002, 9, 443-454; Bryan, M. C. et al., Chem. Biol. 2002, 9, 713-720; Sun, X. L. et al., J. Am. Chem. Soc. 2002, 124, 7258-7259; Sackmann, E., Science 1996, 271, 43-48.)

Fundamental studies of glycopolymer properties have provided insight regarding carbohydrate-mediated biomolecular recognition processes that may be attributed, in part, to a multivalent or cluster effect. (Bovin, N. V. and Gabius, H. J., Chem. Soc. Rev. 1995, 413-421; Roy, R., Trends Glycosci. Glycotechnol. 1996, 8, 79-99; Kiessling, L. L. et al., Curr. Opin. Chem. Biol. 2000, 4, 696-703; Bertozzi C. R. and Kiessling, L. L., Science 2001, 291, 2357-2364.) Significantly, these efforts hold relevance for both pharmaceutical and biomaterial applications. For example, recent investigations have synthesized glycopolymers with surface anchoring groups located along the polymer backbone to generate glycosurfaces with potential utility in bio- and immunochemical assays (Roy, R. et al., Chem. Soc., Chem. Commun. 1992, 1611-1613; Thoma, G. et al., J Am. Chem. Soc. 1999, 121, 5919-5929) as well as biocapture analysis. (Bundy, J. L. and Fenselau, C. Anal. Chem. 2001, 73, 751-757.)

Polymers with biotinylated end groups have been used to generate self-organizing protein-polymer hybrid amphiphiles (Hannink, J. M. et al., Angew Chem. Int. Ed. 2001, 40, 4732-4735) as well as molecularly-engineered surfaces. (Cannizzaro, S. M. et al., Biotechnol. Bioeng. 1998, 58:529-535. Black, F. E. et al., Langmuir 1999, 15, 3157-3161.) Biotin and streptavidin activation techniques have played an important role in the development of biofunctionalized surface for sensor or biomaterial applications. (Yang, Z. et al., Langmuir 2000, 16:7482-7492; Hyun, J. et al., Langmuir 2001, 17:6358-6367.) Characteristically, these chain-end functionalized polymers are prepared by further modification or conversion in several steps after initial polymer synthesis. (Hawker, C. J. and Hedrick, J. L. Macromolecules 1995, 28:2993-2995; Rodlert, M. et al., J. Polym. Sci. Polym. Chem. 2000, 38:4749-4763; Harth, E. et al., Macromolecules 2001, 34:3856-3862.)

The formation of a stabilized, membrane-mimetic film on a polyelectrolyte multiplayer (PEM) by in situ photopolymerization of an acrylate-functionalized phospholipid assembly at a solid/liquid interface was described in Marra, K. G. et al., Langmuir 1997, 13, 5697-5701; Orban, J. M. et al., Macromolecules 2000, 33, 4205-4212; and Liu, H. et al., Langmuir 2002, 18, 1332-1339.

All patent applications and publications referred to herein are incorporated by reference to the extent not inconsistent herewith.

Polymers useful for biomolecular recognition processes which can be immobilized onto surfaces, and which are easily synthesized are needed.

### SUMMARY OF THE INVENTION

This invention provides polymers, including glycopolymers, useful for biomolecular recognition processes which can be immobilized onto surfaces and which are easily synthesized.

Such polymers are broadly described as molecules comprising a polymer backbone; with pendent multivalent groups attached to the polymer backbone; and an anchoring group attached to the polymer backbone for covalently or noncovalently attaching the molecule to a surface or another molecule.

Such molecules may be represented by the following structural formula:

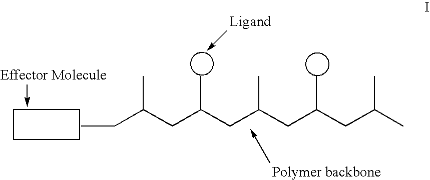

A preferred embodiment is:

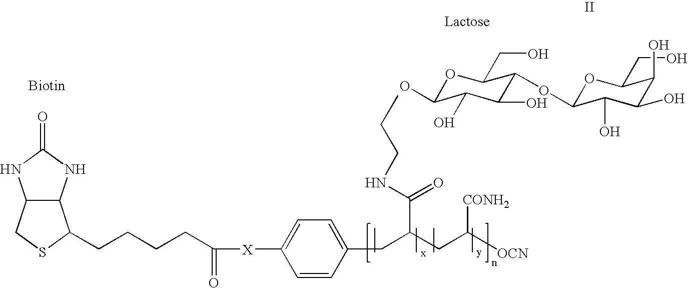

This invention provides a wide range of molecules having anchoring groups, e.g., any moiety that can be biotinylated can be immobilized onto an avidin/streptavidin surface.

The polymer backbone may be straight or branched, saturated or unsaturated. For example, it may be prepared by polymerization of a glycomonomer having a polymerizable functionality such as acryloyl, methacryloyl or vinyl and acrylarnide or methacrylamide.

The pendent multivalent group is preferably a saccharide, such as a monosaccharide, disaccharide, trisaccharide or oligosaccharide. Such saccharides may be sulfated, i.e., one or more OH moieties are replaced by SO₃. Embodiments of this invention include as the saccharide N-acetyl-D-glucosamine, α- and β-N-acetyl-D-glucosamine-(1-4)-β-D-glucuronic acid, pyranosides, lactose, and/or polylactose.

The pendent multivalent group may also be glycosaminoglycan, sialic acid, siayl Liews X, heparin, and/or oligopeptides.

These molecules preferably have a polydispersity index (molecular weight Mw/molecular number Mn) between about 1.1 and about 1.5 and have between about 2 and about 1000 pendent multivalent groups.

The molecules preferably also comprise a spacer arm between the anchoring group and the polymer backbone. The spacer arm may be alkyl, or alkenyl, such as C3-C9, and may comprise a ring, such as an aryl or cycloalkyl ring. The spacer arm may also comprise arylamide, methacrylamide, acryloyl, methacrylol, and vinyl. As is understood by those of skill in the art, the aryl ring (preferably a phenyl ring) or cycloalkyl ring of the foregoing molecules may have additional substituents which do not interfere with use of the compounds as described herein, and hydrogens in the above molecules may be replaced with groups such as methyl, ethyl, other lower alkyl groups, amine, sulfur, nitrogen, phosphorus and other atoms and groups which do not interfere with the use of the compounds as described herein, and oxygen can be substituted for sulfur in the above molecules. Further, the C—C—C—C carbon chains of the anchoring group, e.g., the biotin and biotin-cap molecules, may be longer or shorter, e.g., 2 to 8 carbons, and may contain additional pendent oxygens, amines, lower alkyl or other groups. Such rings may comprise at least one substituent which, depending on the electronic and steric effects of any substituent present, as is known to the art, may be ortho, meta orpara, to the position at which the ring attaches to the polymer backbone. Ring substituents can be hydrocarbyl. Particular substituents are, e.g., methoxy, alcohol, ether, amine, polyamine, sulfate, phosphate, nitrate, nitrite, halogen selected from the group consisting of chlorine, bromine and iodine, salts of the foregoing, and other substituents which do not interfere with formation of the polymer via free radical polymerization. Preferably the substituent is apara substituent. Rings include 5-, 6-, or 7-membered rings, saturated or unsaturated.

The term “hydrocarbyl” is used herein to refer generally to organic groups comprised of carbon chains to which hydrogen and optionally other elements are attached. CH₂ or CH groups and C atoms of the carbon chains of the hydrocarbyl may be replaced with one or more heteroatoms (i.e., non-carbon atoms). Suitable heteroatoms include but are not limited to O, S, P and N atoms. The term hydrocarbyl includes, but is not limited to, alkyl, alkenyl, alkynyl, ether, polyether, thioether, ascorbate, aminoalkyl, hydroxylalkyl, thioalkyl, aryl and heterocyclic aryl groups, amino acid, polyalcohol, glycol, groups which have a mixture of saturated and unsaturated bonds, carbocyclic rings and combinations of such groups. The term also includes straight-chain, branched-chain and cyclic structures or combinations thereof. Hydrocarbyl groups are optionally substituted. Hydrocarbyl substitution includes substitution at one or more carbons in the group by moieties containing heteroatoms. Suitable substituents for hydrocarbyl groups include but are not limited to halogens, including chlorine, fluorine, bromine and iodine, OH, SH, NH, NH₂, COH, CO₂H, ORₐ, SRₐ, NRₐR_{b}, CONRₐR_{b}, where Rₐ and R_{b} independently are alkyl, unsaturated alkyl or aryl groups, sulfate, sulfite, phosphate, nitrate, carbonyl, and polyamine.

The term “amine” refers to a primary, secondary, or tertiary amine group. A “polyamine” is a group that contains more than one amine group. A “sulfate” group is a salt of sulfuric acid. Sulfate groups include —SO₃, the group (SO₄)²⁻ and sulfate radicals. “Phosphates” contain the group PO₄³⁻. “Glycols” are groups that have two alcohol groups per molecule of the compound.

The term “alkyl” takes its usual meaning in the art and is intended to include straight-chain, branched and cycloalkyl groups. The term includes, but is not limited to, methyl, ethyl, n-propyl, isopropyl, n-butyl, sec-butyl, isobutyl, tert-butyl, n-pentyl, neopentyl, 2-methylbutyl, 1-methylbutyl, 1-ethylpropyl, 1,1-dimethylpropyl, n-hexyl, 1-methylpentyl, 2-methylpentyl, 3-methylpentyl, 4-methylpentyl, 3,3-dimethylbutyl, 2,2-dimethylbutyl, 1,1-dimethylbutyl, 2-ethylbutyl, 1-ethylbutyl, 1,3-dimethylbutyl, n-heptyl, 5-methylhexyl, 4-methylhexyl, 3-methylhexyl, 2-methylhexyl, 1-methylhexyl, 3-ethylpentyl, 2-ethylpentyl, 1-ethylpentyl, 4,4-dimethylpentyl, 3,3-dimethylpentyl, 2,2-dimethylpentyl, 1,1-dimethylpentyl, n-octyl, 6-methylheptyl, 5-methylheptyl, 4-methylheptyl, 3-methylheptyl, 2-methylheptyl, 1-methylheptyl, 1-ethylhexyl, 1-propylpentyl, 3-ethylhexyl, 5,5-dimethylhexyl, 4,4-dimethylhexyl, 2,2-diethylbutyl, 3,3-diethylbutyl, and 1-methyl-1-propylbutyl. Alkyl groups are optionally substituted. Lower alkyl groups are C₁-C₆ alkyl and include among others methyl, ethyl, n-propyl, and isopropyl groups.

The term “cycloalkyl” refers to alkyl groups having a hydrocarbon ring, preferably to those having rings of 3 to 7 carbon atoms. Cycloalkyl groups include those with alkyl group substitution on the ring. Cycloalkyl groups can include straight-chain and branched-chain portions. Cycloalkyl groups include but are not limited to cyclopropyl, cyclobutyl, cyclopentyl, cyclohexyl, cycloheptyl, cyclooctyl, and cyclononyl. Cycloalkyl groups can optionally be substituted.

The term “unsaturated alkyl” group is used herein generally to include alkyl groups in which one or more carbon-carbon single bonds have been converted to carbon-carbon double or triple bonds. The term includes alkenyl and alkynyl groups in their most general sense. The term is intended to include groups having more than one double or triple bond, or combinations of double and triple bonds. Unsaturated alkyl groups include, without limitation, unsaturated straight-chain, branched or cycloalkyl groups. Unsaturated alkyl groups include without limitation: vinyl, allyl, propenyl, isopropenyl, butenyl, pentenyl, hexenyl, hexadienyl, heptenyl, cyclopropenyl, cyclobutenyl, cyclopententyl, cyclopentadienyl, cyclohexenyl, cyclohexadienyl, 1-propenyl, 2-butenyl, 2-methyl-2-butenyl, ethynyl, propargyl, 3-methyl-1-pentynyl, and 2-heptynyl. Unsaturated alkyl groups can optionally be substituted.

Substitution of alkyl, cycloalkyl and unsaturated alkyl groups includes substitution at one or more carbons in the group by moieties containing heteroatoms. Suitable substituents for these groups include but are not limited to OH, SH, NH₂, COH, CO₂H, OR_{c}, SR_{c}, P, PO, NR_{c}R_{d}, CONR_{c}R_{d}, and halogens, particularly chlorines and bromines where R_{c} and R_{d}, independently, are alkyl, unsaturated alkyl or aryl groups. Preferred alkyl and unsaturated alkyl groups are the lower alkyl, alkenyl or alkynyl groups having from 1 to about 3 carbon atoms.

The term “aryl” is used herein generally to refer to aromatic groups which have at least one ring having a conjugated pi electron system and includes without limitation carbocyclic aryl, aralkyl, heterocyclic aryl, biaryl groups and heterocyclic biaryl, all of which can be optionally substituted. Preferred aryl groups have one or two aromatic rings.

Aryl groups may be substituted with one, two or more simple substituents including, but not limited to, lower alkyl, e.g., methyl, ethyl, butyl; halo, e.g., chloro, bromo; nitro; sulfato; sulfonyloxy; carboxy; carbo-lower-alkoxy, e.g., carbomethoxy, carbethoxy; amino; mono- and di-lower-alkylamino, e.g., methylamino, ethylamino, dimethylamino, methylethylamino; amido; hydroxy; lower-alkoxy, e.g., methoxy, ethoxy; and lower-alkanoyloxy, e.g., acetoxy.

“Carbocyclic aryl” refers to aryl groups in which the aromatic ring atoms are all carbons and includes without limitation phenyl, biphenyl and naphthalene groups.

“Aralkyl” refers to an alkyl group substituted with an aryl group. Suitable aralkyl groups include among others benzyl, phenethyl and picolyl, and may be optionally substituted. Aralkyl groups include those with heterocyclic and carbocyclic aromatic moieties.

“Heterocyclic aryl groups” refers to groups having at least one heterocyclic aromatic ring with from 1 to 3 heteroatoms in the ring, the remainder being carbon atoms. Suitable heteroatoms include without limitation oxygen, sulfur, and nitrogen. Heterocyclic aryl groups include among others furanyl, thienyl, pyridyl, pyrrolyl, N-alkyl pyrrolo, pyrimidyl, pyrazinyl, imidazolyl, benzofuranyl, quinolinyl, and indolyl, all optionally substituted.

The molecules of this invention may comprise hydrophobic and hydrophilic substituents as is known to the art.

The anchoring group may be any group which facilitates conjugation of the foregoing molecules to surfaces or other molecules, such as any group allowing covalent binding to a binding partner, or noncovalent binding to another molecule or surface. The anchoring group is preferably selected from the group consisting of biotin, biotin-cap, charged anchoring groups such as styrene sulfate, styrene sulfonate, alkyl sulfate, alkyl sulfonate, and alkyl sulfonic acid; chemically-reactive groups such as thiols, amines, aldehydes and carboxylic acids, photocrosslinkable groups such as aryldiazirine and arylazide, and hydrophobic groups such as C₆-C₂₂ alkyl, C₁₂-C₂₂ lipid or C₁₂-C₂₂ phospholipid. The term “lipid” herein includes phospholipids.

The molecule preferably comprises a first pendent group comprising a linking group to connect it to the polymer backbone and a multivalent moiety connected to the linking group. The first pendent unit may comprise a hydrocarbyl-linking group connected to the backbone. Preferably the linking group contains about 3 to about 9 atoms, most preferably carbon atoms. It is preferably (CH₂)ₙ where n is 3 to 9, and preferably is a straight chain, but may also be an unsaturated and/or branched chain, may comprise additional moieties attached to the chain, and the chain may comprise heteroatoms, so long as formation of the polymer via free radical polymerization is not interfered with by such heteroatoms, branching, and/or substituents. When the first pendent unit is made using an alkenyl-based glycomonomer, this helps lower the polydispersity index, thus it is preferred that an alkenyl-based glycomonomer be used.

The molecule may also comprise a second pendent unit. The second unit consists of or comprises a lipid or long alkyl chain of about C₇ to C₃₀. Preferably the second pendent unit comprises about 3 to about 9 atoms. It may comprise substituents, branches, and hetero-atoms as does the linking group. It is preferably formed of an easily polymerizable hydrocarbyl monomer by copolymerization along with the first unit. Preferably the second unit is polymerized from acrylamide, acrylate, or alkenyl compounds, most preferably acrylamide. The molecules of this invention may comprise up to about 1000 of such second units. The second unit may function as a spacer, or it may be derived from an easily-polymerizable monomer that promotes polymerization during the process of making the molecules of this invention.

The first and second units may be interspersed with each other in random order along the polymer backbone. As is known to the art, polymerization process parameters such as concentration of reactants, and temperature, will determine the statistical frequency and order of the first and second units.

The number of pendent units may be up to about 2000. In one embodiment, the second pendent unit comprises or consists of phosphatidylcholine. When a second pendent unit is present, the sum of the number of multivalent moieties and second unit moieties is preferably no more than about 2000, more preferably no more than about 1000, and most preferably no more than about 100. The first and second units maybe in cis- or trans- configuration with respect to each other. Consecutive first units may also be cis- or trans- to each other.

The first and second units may be interspersed with each other in random order along the polymer backbone. As is known to the art, polymerization process parameters, such as concentration of reactants and temperature, will determine the statistical frequency and order of the first and second units.

In certain embodiments of this invention, the molecule comprises a cyanoxyl group at the end of the polymer backbone opposite the anchoring group.

These molecules are useful for providing surfaces capable of binding to bioactive molecules for use in bio and immunochemical assays, as well as biocapture analysis. Bioactive molecules can include thrombomodulin, growth factors, cytokines, proteins, peptides, cells, viruses, antibodies, fluorescent groups, FAB fragments, IgG, catalysts, ligand molecules for capture of other molecules, etc., as is known to the art. These molecules may also be used to target specific bioactive molecules in drug delivery methods. Activated surfaces, comprising such bioactive molecules bound to the multivalent groups of the molecules of this invention, are also provided herein.

This invention also provides a bioactive surface comprising the foregoing molecules covalently or non-covalently bound to the surface via the anchoring group. Such surfaces may be any surface known to the art to which it is desired to impart biological functional properties, e.g., metal, glass, silicon, synthetic polymers, natural polymers such as alginate and collagen, surfaces of medical devices in contact with blood or tissue such as vascular grafts, catheters, and biosensors, and membrane mimetic surfaces. PCT publications WO98/16198, WO 00/00239, WO 01/78800, WO 02/055021 and WO 02/09647, and U.S. application Ser. No. 60/428,438 describe membrane-mimetic materials and polymers useful for binding to bioactive molecules which are useful in the practice of this invention. All these reference are incorporated herein by reference to the extent not inconsistent herewith. Definitions of terms used in said references apply hereto unless application of such definitions would result in inconsistency or loss of meaning, in which case, explicit or implicit definitions used herein apply to the disclosure hereof. Such membrane-mimetic surfaces include glycocalyx-mimetic surfaces, supported lipid surfaces, and polyelectrolyte multilayers. A supported lipid surface is defined as a single continuous phospholipid bilayer on a solid or polymeric surface. A polyelectric multilayer is defined as a surface made by electrostatic self-assembly of polyelectrolytes, also referred to as layer-by-layer assembly of polyelectrolytes . In one embodiment, the surface is made up of an elastic polypeptide block copolymer having hydrophilic and hydrophobic groups.

Surfaces for binding to the molecules of this invention can be prepared by immobilizing a binding partner for an anchoring group of this invention onto a solid polymeric surface by covalent or non-covalent binding. Examples are acid- or Biotin-modified PET surfaces, or biotin, biotin-lipid modified silicon, glass or medical device surfaces.

This invention also comprises the foregoing molecules covalently bound via their anchoring groups to appropriate binding partners such as avidin, streptavidin, an amine-containing group which links via —OCN by isourea bond formation such as a protein, a polypeptide, a polymer, a dendrimer, or a lipid.

In one embodiment, two, three or four of the molecules of this invention may be bound to the same binding partners via their anchoring groups to make dimers, trimers, and tetramers. Such complexes are useful for binding to bioactive molecules such as antigens, to provide a higher cluster density of antigenic pendent moieties, to improve antigenicity and circulating half-life. Such molecules are also useful to carry targeting groups attached to the multivalent portions of one or more portions of the molecule and other bioactive molecules such as toxins bound to other multivalent portions of the molecule.

The molecules of this invention may also be covalently or non-covalently bound to a biological marker known to the art such as an organic or inorganic fluorescent dye or nanoparticle.

Sulfated saccharide molecules of this invention are useful for a variety of biological purposes.

One embodiment of this invention provides a sulfated molecule designated SL3 having a molecular weight of about 9300 and a PDI of 1.46 which is a copolymer of sulfated lactose monomers and acrylamide at a molar ratio of acrylamide to sulfated lactose monomers of about 1:10 and a lactose content of about 57 wt %.

Such molecules are as effective as heparin in preventing coagulation of blood. Thus, this invention comprises reducing coagulation of blood by contacting the blood with an effective amount of a sulfated glycopolymer of this invention, preferably SL3.

These sulfated molecules are also useful for promoting binding of growth factors to their receptors and stimulating cell proliferation when an effective amount of such sulfated molecules, preferably heptasulfate disaccharides, and more preferably SL3, are placed in contact with cells which are responsive to growth factors. The sulfated molecules also selectively stimulate FGF-2 growth factor to its receptor, i.e., they do not stimulate FGF-1 binding.

The sulfated polymer molecules of this invention are also useful for preventing degradation, under conditions of heat, low pH and proteolytic enzymes, of soluble growth factors released into extra-cellular matrix when an effective amount of such sulfated polymer molecules, preferably sulfated saccharide molecules, and more preferably SL3, are placed in contact with growth factors such as FGF-2.

This invention also provides molecules having the following formula: 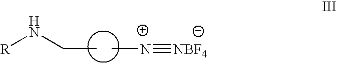
wherein R is an anchoring group, and o represents a ring selected from the group consisting of aryl, aralkyl, cycloalkyl, cycloalkenyl, heterocyclic rings and substituted rings.

This invention further provides molecules having the following formula: 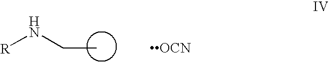
wherein R is an anchoring group, and o represents a ring selected from the group consisting of aryl, aralkyl, cycloalkyl, cycloalkenyl, heterocyclic rings and substituted rings.

This invention further provides molecules having the following formula: 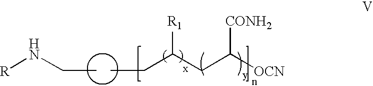
wherein R is an anchoring group; o represents a ring selected from the group consisting of aryl, aralkyl, cycloalkyl, cycloalkenyl, heterocyclic rings and substituted rings; x is between about 1 and about 50, preferably between about 5 and about 30 and more preferably between about 5 and about 12; y is between about 1 and about 100, preferably between about 50 and about 80; n is between about 1 and about 100, preferably between about 10 and 50; R1 is selected from the group consisting of monosaccharides, oligosaccharides, polysaccharides and glycosaminoglycans such as galactose, sialic acid, siayl Liews X, heparin, oligopeptides and other biomolecules, conjugated to —(CH2)m-HN—CO— which is conjugated to the polymer backbone; and wherein m is 1 to about 5.

This invention also provides methods of making the foregoing molecules comprising the steps of:
(a) providing a compound having the structure: 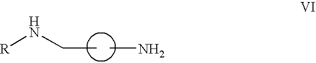
   wherein R is an anchoring group; and
   ◯represents a ring selected from the group consisting of aryl, aralkyl, cycloalkyl, cycloalkenyl, heterocyclic rings and substituted rings;
(b) reacting said molecule of step (a) with HBF₄ to form a molecule having the structure: 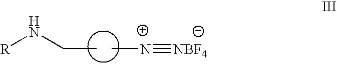
(c) reacting the molecule of step (b) with a cyanate to form a molecule having the structure: 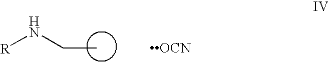
(d) reacting the molecule of step (c) with a molecule having a multivalent moiety and a terminal vinyl group to form a compound having the formula: 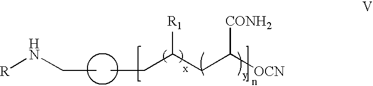
   where x is between about 1 and about 50, preferably between about 5 and about 30, and more preferably between about 5 and about 12;
   y is between about 1 and about 100, preferably between about 50 and about 80;
   n is between about 1 and about 100, preferably between about 10 and 50;
   and R₁ is selected from the group consisting of monosaccharides, oligosaccharides, polysaccharides and glycosaminoglycans such as galactose, sialic acid, siayl Liews X, heparin, oligopeptides and other biomolecules, conjugated to —(CH₂)m-HN—CO— which is conjugated to the polymer backbone; and
   wherein m is 1 to about 5.

The reaction mixture of step (d) may also include a comonomer comprising a vinyl group. The comonomer may be acrylamide, acrylate, C₁-C₁₀ alkenyl, and their derivatives. Derivatives include such groups having substituents which do not interfere with the reaction.

This invention also provides methods of growing the polymers of this invention having multivalent substituents directly on a surface by first attaching a molecule of Formula VI, VII or VIII to the surface and conducting the polymerization reactions in: situ.

The molecules of this invention comprising multivalent groups may be covalently bound to bioactive moieties such as proteins, e.g., growth factors and cytokines, as well as viruses, cells and substrates, through the multivalent groups, as is known to the art with respect to carbohydrate-mediated biomolecular recognition processes. The end-product molecules of this invention are useful in protein separation, cell culture, and drug-delivery systems, as well as in targeting for treatment of wound healing and other pathological conditions.

These molecules are useful for providing surfaces capable of binding to bioactive molecules for use in bio and immunochemical assays, as well as biocapture analysis. Bioactive molecules can include thrombomodulin, growth factors, cytolines, proteins, peptides, cells, viruses, antibodies, fluorescent groups, FAB fragments, IgG, catalysts, ligand molecules for capture of other molecules, etc., as is known to the art. These molecules may also be used to target specific bioactive molecules in drug delivery methods. Surfaces with the foregoing molecules bound thereto, and also with bioactive molecules attached to the multivalent moieties of said molecules are also provided herein.

### BRIEF DESCRIPTION OF THE FIGURES

FIG. 1 is an ¹H NMR spectrum of the biotin terminated glycopolymer of Formula II.

FIG. 2 shows results of a streptavidin-biotin binding SDS-PAGE gel shift assay: (1) two equivalents of glycopolymer; (2) 20 equivalents of glycopolymer. A, marker; C, Streptavidin alone; D, streptavidin plus p-chlorophenyl-glycopolymer; E, streptavidin plus biotin-glycopolymer of Formula II; F, streptavidin plus biotin-cap-glycopolymer of Formula II with biotin-cap substituted for biotin.

FIG. 3 is a schematic representation of lectin binding to a glycopolymer-derivatized surface.

FIG. 4 is a schematic representation of a glycocalyx-mimetic thin film produced by coupling biotin-terminated glycopolymers to a supported lipid membrane.

FIG. 5 shows advancing and receding contact angles of successive assemblies of an alginate/poly-L-lysine (ALG/PLL) multilayer, terpolymer (TER), biotin-PE/PC lipid vesicles (B-PE/PC), streptavidin (STREP) and glycopolymer (GLY): FIG. 5A shows results for 10 mol % biotin-PE; FIG. 5B shows results for 25 mol % biotin-PE, and FIG. 5C shows results for 50 mol % biotin-PE.

FIG. 6 shows parallel (Rp)(I) and perpendicular (Rs)(II) polarized external reflection infrared spectra of polymerized films: 10 mol % biotin-PE/PC (FIG. 6A) 25 mol % biotin-PE/PC (FIG. 6B); and 50 mol % biotin-PE/PC (FIG. 6C). IR bands labeled with an asterisk in Rp polarized spectra indicate CH₂ absorption modes that are changing in intensity with increasing biotin-PE content as shown in part I, spectra A-C.

FIG. 7 shows parallel (Rp) (I) and perpendicular (Rs) (II) polarized external reflection infrared spectra of a polymerized 10 mol % biotin-PE/PC film (FIG. 7A); spectra following successive absorption of streptavidin (FIG. 7B); and a glycopolymer (FIG. 7C). IR bands labeled with an asterisk in Rp polarized spectra indicate CH₂ absorption modes that are changing in intensity after streptavidin binding to the biotin-PE-functionalized surface as shown in part I, spectra A and B.

FIG. 8 shows parallel (Rp) (I) and perpendicular (Rs) (II) polarized external reflection infrared spectra of a polymerized 25 mol % biotin-PE/PC film (FIG. 8A); spectra following successive absorption of streptavidin (FIG. 8B) and a glycopolymer (FIG. 8C). IR bands labeled with an asterisk in Rp polarized spectra indicate CH₂ absorption modes that are changing in intensity after streptavidin binding to the biotin-PE-functionalized surface as shown in part I, spectra A and B.

FIG. 9 shows parallel (Rp) (I) and perpendicular (Rs) (II) polarized external reflection infrared spectra of a polymerized 50 mol % biotin-PE/PC film (FIG. 9A); spectra following successive absorption of streptavidin (FIG. 9B), and a glycopolymer (FIG. 9C).

FIG. 10 shows PTT dose-response study of glycopolymers bearing sulfated lactose units. Both Lovenox (LMW heparin) and heparin are included as positive controls. Significant prolongation of partial thromboplastin time (PTT) is observed with SL3, a sulfated lactose polymer of this invention.

FIG. 11 shows [³H]thymidine incorporation into BaF3-FR1C-11 cells treated with 3 ng/mL of growth factor FGF-2 and increasing concentrations of heparin or indicated test compounds.

FIG. 12 shows the effect of heparin or sulfated lactose polymer SL3 (25 μg/mL) on the biological activity of heat-treated basic FGF.

### DETAILED DESCRIPTION

Polymers and molecules of this invention comprise anchoring groups as means for binding to a surface to form a membrane-mimetic surface. Avidin (streptavidin)/biotin binding provides preferred means for binding said molecules. The molecules, polymers and surfaces of this invention have the advantage of being rapidly synthesized in a mild aqueous environment with simple washing and purification steps. Thus, potential damage to candidate surface ligand groups due to the conjugation process is limited.

This invention also provides stable, substrate-supported model membranes. Preferred membranes emulate the structural features of the glycocalyx. These model membranes and surface modification strategies are useful to enhance the functionality of biosensors, biochips, and microfluidic devices, and to improve the clinical performance characteristics of blood-contacting artificial organs and other implantable medical devices by modulating maladaptive processes at the blood-material and tissue/material interfaces.

Membrane-bound carbohydrates can exert a steric repulsive effect that improves the specificity of biological interactions by limiting nonspecific cell/cell and protein/cell binding. Nonetheless, glycopolymers bearing a series of pendent anchoring groups characteristically demonstrate reduced bioactivity due to steric hindrance, which can be partially offset through the introduction of a spacer arm between the anchor and the polymer backbone.

Preferred embodiments of this invention are further described below. As will be apparent to those skilled in the art, other molecules, substituents, and reagents may be substituted for those disclosed herein to provide the molecules and surfaces as broadly described hereinabove.

The molecules in solution are in equilibrium with free cyanoxyl radicals which may leave the terminal end of the polymer backbone, leaving it free to participate in further polymerization reactions.

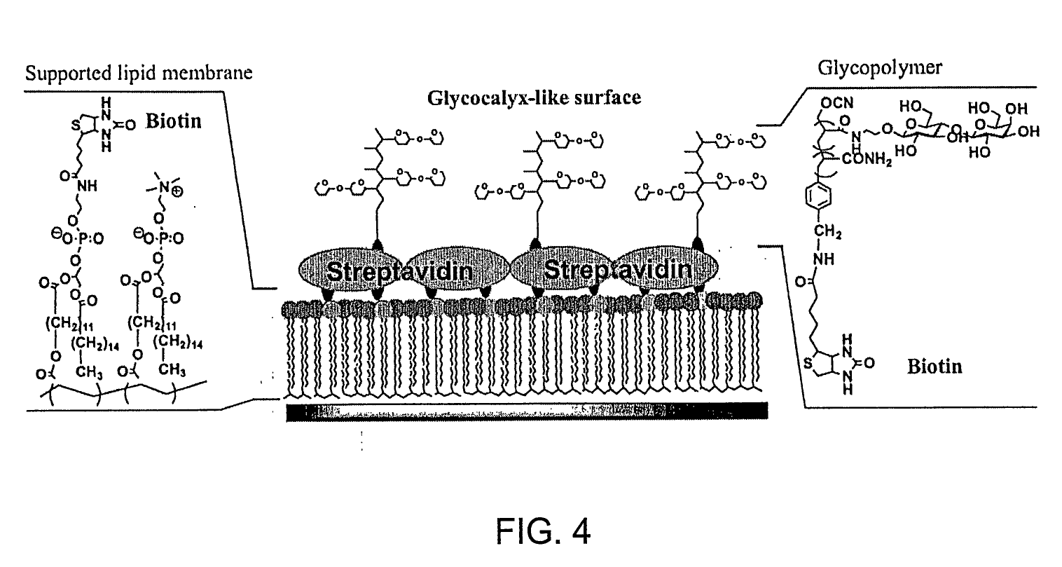

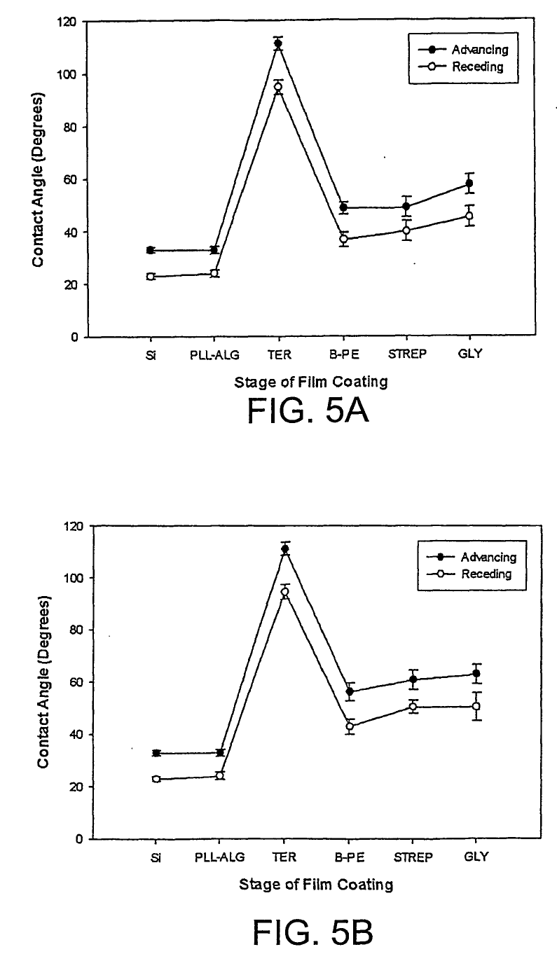

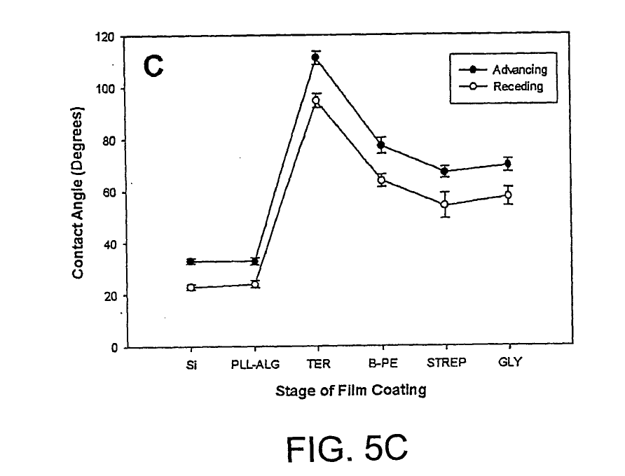

In one embodiment of the present invention, biotin chain-terminated polymers were synthesized bearing pendent lactose units. These glycopolymers were used to fabricate a glycocalyxlike structure on a membrane-mimetic film composed of mixed polymerizable lipids containing phosphatidylcholine or biotin headgroups (FIG. 4). Polarized external reflection infrared spectroscopy in combination with confocal fluorescence microscopy was utilized to characterize the detailed molecular structure of this system and the uniformity of the thin film coating after streptavidin and glycopolymer binding. Biotinylated lipids appear to cluster in microdomains within the membrane-mimetic film, particularly at high surface density. Nonetheless, a uniform carbohydrate coating was achieved after glycopolymer binding to the membrane-mimetic film.

In another embodiment, molecules of this invention were covalently bonded to a synthetic protein copolymer having selected plastic and elastic properties as described in U.S. Patent Application Ser. No. 60/428,438 filed Nov. 22, 2002, incorporated herein by reference to the extent not inconsistent herewith. The surface is made of a protein copolymer comprising at least one hydrophilic block and at least one hydrophobic block, and up to three or more blocks. In one embodiment, the surface comprises a protein copolymer having a first end block, a second end block, and a middle block, wherein said first and second end blocks are substantially identical. In a preferred embodiment, the protein copolymer comprises hydrophobic end blocks and a hydrophilic middle block.

In a particular embodiment of this invention, the surface is made of an elastic polypeptide copolymer comprising a first and last end block which comprises a polypeptide encoded by a nucleic acid sequence of [VPAVG(IPAVG)₄]ₙ or a [(IPAVG)₄(VPAVG)]ₙ sequence, and the middle block comprises a polypeptide encoded by a nucleic acid sequence selected from the group consisting of: [(VPGEG)(VPGVG)₄]ₘ, [(VPGVG)₄(VPGEG)]ₘ, and (VPGVG)₂VPGEG(VPGVG)₂]ₘ. In another embodiment, the surface is made of a protein copolymer which comprises endblocks selected from amino acid sequences encoded by the above endblock nucleotide sequences and a middle block selected from amino acid sequences encoded by the above middle block nucleotide sequences, n is from about 5 to about 100, and m is from about 10 to about 100. In a particular embodiment, n is about 16.

In another embodiment, the middle block is a polypeptide selected from the group consisting of polypeptides encoded by:

VPGVG [VPGVG(VPGIGVPGVG)₂]₁₉VPGVG;

VPGVG [(VPGVG)₂VPGEG(VPGVG)₂]₃₀VPGVG;

VPGVG [(VPGVG)₂VPGEG(VPGVG)₂]₃₈VPGVG;

VPGVG [(VPGVG)₂VPGEG(VPGVG)₂]₄₈VPGVG;

VPGVG [VPGVG(VPNVG)₄]₁₂VPGVG;

VPGVG [(APGGVPGGAPGG)₂]₂₃VPGVG;

VPGVG [(APGGVPGGAPGG)₂]₃₀VPGVG;

[VPGVG(IPGVGVPGVG)₂]₁₉;

[VPGEG(VPGVG)₄]₃₀;

[VPGEG(VPGVG)₄]₄₈;

[(APGGVPGGAPGG)₂]₂₂; and

[(VPGMG)₅]ₓ,

wherein x is from about 10 to about 100.

These protein copolymers have elastic properties, i.e., are able to stretch from about 2.5 up to about 14 times their initial length.

Surfaces may be derivatized with binding groups such as streptavidin, which binds to biotin, by methods described in Example 2 below. Surfaces may also be prepared for binding to the molecules of this invention by derivatizing with other binding groups known to the art which bind to specific anchoring groups used in this invention.

A straightforward approach is provided to synthesize biotin chain-terminated glycopolymers of low polydispersity (Formulas I and II), via use of a biotin-derivatized arylamine initiator employed in a cyanoxyl-mediated free radical polymerization scheme.

### EXAMPLE 1

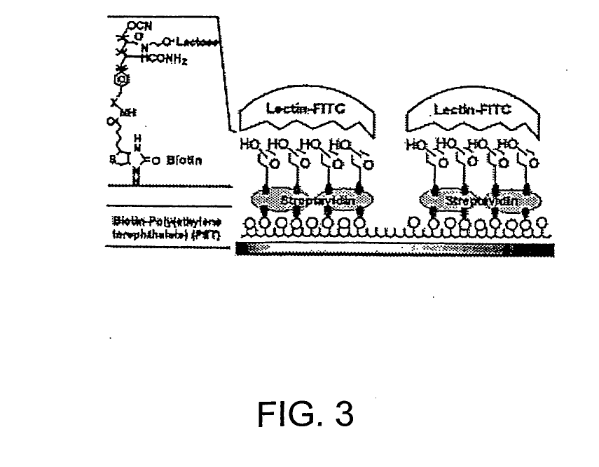

A representative chain end-functionalized glycopolymer was designed as exemplified in Formula II and FIG. 3 in which multivalent lactose units serve as ligands for lectins and/or antibodies, and a single biotin group provides an anchor by its specific binding activity to avidin or streptavidin. Modulating lactose density as well as polymer solubility was achieved by using acrylamide as a comonomer and a spacer arm (X) between biotin and the polymer backbone was used for further optimization of the polymer-avidin/streptavidin interaction. The synthetic strategy relies on our previously developed cyanoxylmediated free-radical polymerization process, in which arylamines have been identified as candidate initiators. (Grande, D. et al., Macromolecules 2000, 33, 1123-1125; Grande, D. et al., Macromolecules 2001, 34, 1640-1646.) Specifically, the biotin derivatized arylamine was used as an initiator for cyanoxyl-mediated free radical polymerization of 2-acrylaminoethyl lactoside and acrylamide so as to provide the desired biotin chain-terminated glycopolymers for subsequent use in glycosurface engineering.

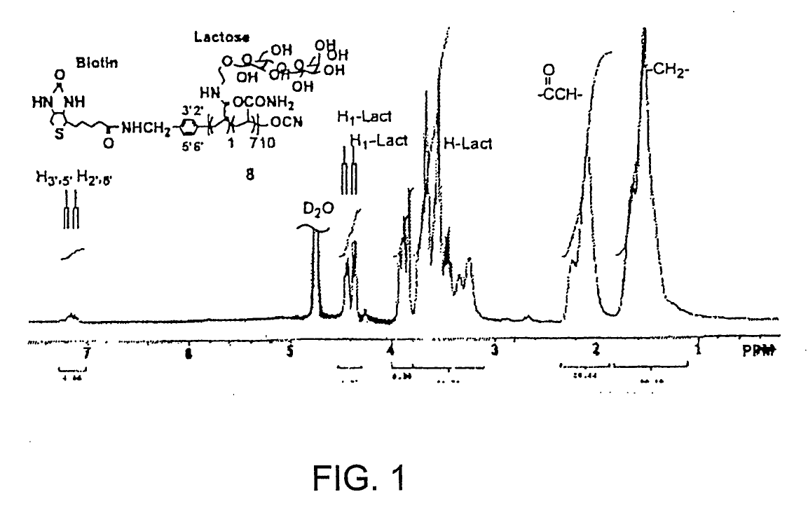

In the current study, 4-aminobenzyl-biotinamide was used wherein the biotin was biotin or biotin-cap: 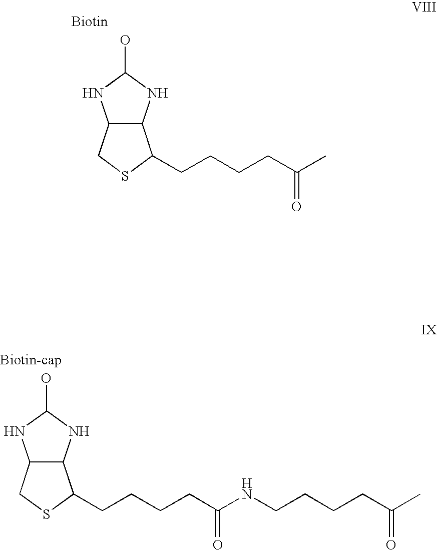
These compounds were designed and investigated as initiators for the polymerization of 2-acrylaminoethyl lactoside with acrylamide. The biotin-containing arylamine initiator 4-aminobenzyl biotinamide was prepared by the condensation of commercial available p-nitrobenzylamine with N-hydroxysuccinimidyl-biotin, and N-hydroxy-succinimidyl-biotinamidocaproate followed by hydrogenation with Pd-C in methanol in high yield, respectively. 2-Acrylaminoethyl lactoside was prepared from lactose per-acetate via four steps including glycosylation, hydrogenation, and acrylation with acryloyl chloride in 53% total yield.) Treatment of the 4-aminobenzyl-biotinamide with HBF4 and NaNO2 in deoxygened H2O-THF (1:1) gave the arenediazonium cation: 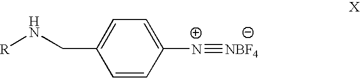
which upon reaction with NaOCN at 50° C. afforded the biotinyl aryl free radical: 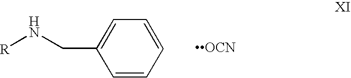
and the cyanoxyl free radical (.OCtN) as the initiating system for copolymerization of glycomonomer 2-acrylaminoethyl lactoside and acrylamide. The biotin chain-terminated glycopolymer: 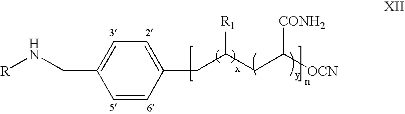
was generated in 75% conversion as a white spongy powder. Similarly, using biotin-cap-arylamide as the initiating species, a glycopolymer was generated with a spacer arm between biotin and the polymer chain in 70% conversion. The resultant copolymers were characterized by NMR spectroscopy (FIG. 1), as well as by size-exclusion chromatography (SEC) coupled with both refractive index and laser-light scattering (LLS) detectors. Comparison of the integrated signal from the phenyl protons (7.10 ppm, H2′, 6′ and 7.18 ppm, H3′, 5′) with that due to the anomeric protons of lactose (4.36 ppm, H1′-Lact and 4.43 ppm, H1′-Lact), as well as that of the polymer backbone methine (2.10 ppm, CH) and methylene (1.55 ppm CH₂), indicated an average polymer composition of 10 lactose and 70 acrylamide units. Notably, downfield shifts of H2′, H6′ phenyl protons (7.10 ppm) demonstrated C—C bond formation between the phenyl group and the polymer backbone. The actual molar mass (Mn) was 12 kDa with a polydispersity index (Mw/Mn) of 1.3 from SEC/RI/LLS for the compound of Formula XIII where R is biotin.

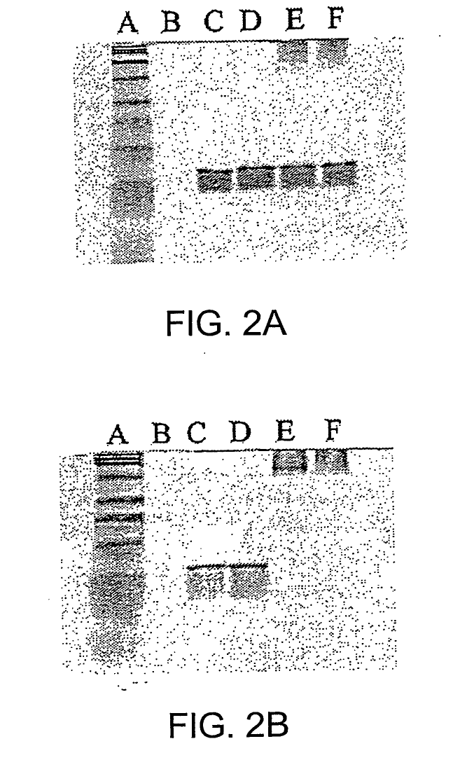

A gel shift assay was performed to verify streptavidin-glycopolymer binding in a solution-phase system. Streptavidin-glycopolymer complexes were generated as illustrated in lanes E and F (FIG. 2, Gel I) while not observed on incubation with a comparable glycopolymer lacking a biotin group [lane D]. Since each streptavidin (60 kDa) contains four identical subunits, a streptavidin band remains present when mixed with only 2 equivalents of biotin-glycopolymer (Gel I, Lanes E and F), but disappears in the presence of an excess of glycopolymer (Gel II, Lanes E and F). The retarded migration of streptavidin-glycopolymer complexes may be due to both an increase in molecular weight and a reduction in the capacity of the apolar portions of streptavidin to interact with the alkyl moiety of sodium dodecysulfate (SDS). (Weber, K. and Osborn, M., J Biol. Chem. 1969, 244, 4406-4412.) In this system, the presence of a spacer arm did not have a measurable impact on glycopolymer/streptavidin affinity.

Glycopolymer-coated surfaces (FIG. 3) were produced by incubating streptavidin-derivatized PET membranes in a glycopolymer solution (1 mg/mL in PBS) for 1 h at room temperature. Membranes were subsequently washed with PBS and incubated in a solution of a FITC-labeled galactose binding lectin (1 mg of psophocarpus tetragonolobus/mL in PBS). Lectin binding was observed in regions of glycopolymer immobilization, while no such activity was noted in surface regions not derivatized with streptavidin.

Cyanoxyl-mediated free-radical polymerization with a biotin-derivatized arylamine initiator has been shown to provide a straightforward strategy for generating biotin chain-terminated glycopolymers. Streptavidin-biotin binding was verified by a SDS-PAGE gel shift assay and the fabrication of a glycocalyx-mimetic surface achieved.

### EXAMPLE 2

Further tests of fabrication of glycocalyx-mimetic surfaces were performed using a biotin chain-terminated glycopolymer which was incorporated onto streptavidin functionalized polymeric lipid films. By variation of carbohydrate type and density, the production of structurally heterogeneous surfaces is facilitated. Significantly, increasing the surface concentration of biotinylated lipids was associated with some increase in hydrocarbon chain disorder and the formation of biotin microdomains. Nonetheless, the size and hydrophilicity of the bound glycopolymer chains led to the generation of a uniform carbohydrate surface coating on a membrane-mimetic thin film.

A glycocalyx-mimetic film was created by the assembly of biotin chain terminated glycopolymers onto a polymeric lipid membrane electrostatically coupled to a polyelectrolyte multilayer. Varying molar compositions (10, 25, and 50 mol %) of acrylate-derivatized biotin-phosphoethanolamine/phosphorylcholine lipid mixtures were prepared as unilamellar vesicles, fused onto the alkyl chains of an amphiphilic terpolymer, and photopolymerized in situ as a planar assembly. Polarized external reflection infrared spectroscopy confirmed the presence of streptavidin. Notably, IR spectroscopy revealed an increase in the conformational and orientational disorder of the lipid hydrocarbon chains with increasing mole fraction of biotinylated lipid. Correlative images obtained by confocal fluorescence microscopy demonstrated that biotinylated lipids cluster at high surface density. Despite the presence of biotin microdomains, the size and hydrophilic characteristics of coupled glycopolymer chains produced a uniform carbohydrate surface coating.

Reagents:

All starting materials and synthetic reagents were purchased from commercial suppliers unless otherwise noted. Poly-L-lysine (PLL, approximately 400 kDa) was purchased from Sigma. Alginate (ALG; low viscosity, ca. 60% mannuronic acid) was obtained from Pronova Biomedical Norway) and used as received. Streptavidin and fluorescein isothiocyanate (FITC) labeled streptavidin were purchased from Calbiochem (San Diego, Calif.). FITC-labeled lectin (from Psophocarpus tetragonolobus) with binding specific to the galactose residues of the glycopolymer was purchased from Sigma. The synthesis and characterization of a terpolymer (TER) composed of 2-hydroxyethyl acrylate (HEA), sodium styrene sulfonate (SSS), and N,N-dioctadecylcarbamoylpropionic acid (DOD), poly(HEA-SSS-DOD) 6:3:1, 1-palmitoyl-2-(12-(acryloyloxy)dodecanoyl)-sn-glycero-3-phosphorylcholine (mono-acrylPC), and 1-palmitoyl-2-(12-(acryloyloxy)dodecanoyl)-sn-glycero-3-phosphorylcholine (mono-acryIPC), and 1-palmitoyl-2-(12-acryloyloxy)dodecanoyl)-sn-glycero-3-phosphoethanolamine (mono-acrylPE) and its biotin derivative (mono-acrylPE-biotin) have been described in detail elsewhere. (Liu, H. et al., Langmuir 2002, 18, 1332-1339; Marra, K. G. et al., Macromolecules 1997, 30, 6483-6488; Chon, J. H. et al., J. Biomater. Sci., Polym. Ed. 1999, 10, 95-108; Sells, T. D. and O'Brien, D. P., Macromolecules 1994, 27, 226-233; Sun, X.-L. et al., Bioconjugate Chem. 2001, 12, 673-677.)

The biotin chain-terminated glycopolymer was generated by cyanoxyl-mediated free radical polymerization, as in Example 1. Briefly, 4-aminobenzyl-biotinamide was used as the initiator for the polymerization of 2-acrylaminoethyl lactoside with acrylaride. Treatment of the initiator with HBF₄ and NaNO₂ in deoxygened H₂O-THF (1:1) generated an arenediazonium cation, which upon reaction with NaOCN at 50° C. afforded a biotinyl-aryl free radical and a cyanoxyl free radical (.OCtN) as the initiating system. A biotin chain-terminated glycopolymer was generated in 75% conversion as a white spongy powder. The molar mass (Mn) of the polymer was 12 kDa with a polydispersity index (Mw/Mn) of 1.3 determined from size-exclusion chromatography coupled with both refractive index and laser-light scattering detectors. The mole fraction of the lactose-bearing repeat units in the biotinylated glycopolymer was 1:7 lactose/acrylamide with a total of 10 repeat units.

Instrumentation:

Contact angles were obtained using a Rame-Hart goniometer, model 100-00. Measurements are reported as the average value plus or minus standard deviation of advancing or receding contact angles of at least 15 data points (5 measurements each per 3 samples).

Infrared Spectroscopy.

Spectra were acquired using a Digilab/BioRad FTS-4000 Fourier transform infrared (FT-IR) spectrometer (Randolf, Mass.) equipped with a wide-band MCT detector, collected with 512 background scans, triangular apodization, and 4 cm⁻¹ resolution. Polarized infrared external reflection spectra were acquired using a Thermo Spectra-Tech 510 external reflection accessory (60° angle of incidence; Shelton, Conn.) with infrared (IR) radiation polarized perpendicular or parallel to the surface normal using a Thermo Spectra-Tech ZnSe wire grid polarizer. Perpendicular (Rs) and parallel (Rp) polarized external reflection spectra were acquired using the spectrum of a clean silicon wafer as a background. Samples were acquired with 300-450 sample scans depending on the amount of water vapor present in the sample spectrum. Reference spectra of biotin-acrylate-PE and acrylate-PC bulk powders were obtained with a Specac Silvergate (Smyrna, Ga.) single bounce attenuated total reflection anvil press accessory. Conditions were similar to those described above, with the exception that the spectra were obtained in an unpolarized format and with 100 background and sample scans. Spectral manipulations performed on the data, such as baseline correction, CO₂ peak removal (from 2250 to 2405 cm⁻¹), and center-of-gravity frequency position determination of IR absorption bands, were performed using the Grams/32 software package (Thermo Galactic Industries, Salem, N.H.). Infrared band assignments were obtained from reference values previously reported in the literature. (Jackson, M. et al., Q. Rev. Biophys. 1997, 30, 365-429; Goormaghtigh, E.; Cabiaux, V.; Ruysschaert, J.-M. In Subcellular Biochemistry, Volume 23: Physicochemical Methods in the Study of Biomembranes; Ralston, H. J. H. a. G. B., Ed.; Plenum Press: New York, 1994; pp 329-362; Shriner, R. L., Fuson, R. C., Curtin, D. Y., and Morrill, T. C. The Systematic Identification of Organic Compounds; John Wiley & Sons: New York, 1980.)

Confocal Fluorescence Microscopy.

Fluorescence images were acquired using a ZeissLSM510 epifluorescence microscope under 63× (oil) magnification and illuminated using an argon ion laser at 485 nm. Lower magnification images acquired in air look similar, albeit with less detail compared to those obtained using oil immersion. The oil was added to the film surface immediately prior to the initiation of imaging. Images were acquired using eight co-added scans in line mode for films coated onto silicon substrates. No post-image processing was performed.

Preparation of Silicon Substrates:

Prime grade silicon wafers were purchased from Si-Tech Inc. (Topsfield, Mass.), were 4 in. in diameter, type P/b (100), with a 4-8 Ω cm resistivity and 500-550 μm thickness, and were polished on one side. Slides were cut using a diamond-tipped glass cutter in dimensions of approximately 1.0 in.×1.5 in. for infrared external reflection spectroscopy and 0.5 in.×0.5 in. for confocal fluorescence microscopy. After cutting, the slides were cleaned by sonication in a 1:8 mixture of Multi-Terge detergent:DI water for 15 min followed by rinsing the samples 10 times in deionized water. The sonication and rinsing procedure was then repeated twice using deionized water alone.

Fabrication of Biotin-Functionalized Supported Lipid Membranes:

Preparation of a (PLL-ALG)5-PLL Terpolymer Film on Silicon.

PLL and ALG were prepared at concentrations of 0.10 and 0.15 w/v % in phosphate-buffered saline (PBS; 20 mM NaH₂PO₄, 0.9 w/v % NaCl, pH 7.4), respectively. PLL and ALG alternating monolayers were deposited on silicon using 60 s contact times for each solution, followed by three rinses with deionized water (˜10 s/rinse) between each coating solution. The (PLL-ALG)5-PLL-coated silicon substrates were then exposed to a 0.1 mM solution of poly(HEA-DOD-SSS) 6:3:1 dissolved in a mixture of 20 mM NaH₂PO₄/DMSO (99:1 v/v), pH) 7.41, for 90 s. The (PLL-ALG)5-PLL terpolymer coated samples were then rinsed 7-10 times with deionized water.

Vesicle Fusion.

Large unilamellar vesicles (LUVs) totaling 12 mM lipid (in either 0, 10, 25, or 50 mol % biotin-mono-acrylPE/mono-acrylPC mixtures) in 20 mM sodium phosphate buffer (pH 7.4) were prepared by three successive freeze/thaw/vortex cycles using liquid N₂ and a 65° C. water bath. The LUVs were then extruded 21 times each through 2.0 μm and 600 mm polycarbonate filters (Millipore), and the solution was diluted to 1.2 mM with 20 mM sodium phosphate buffer (pH 7.4) and 750 mM NaCl. The (PLL-ALG)5-PLL terpolymer-coated substrates were then incubated with the vesicle solution at 43° C. overnight for 14-16 h.

In Situ Photopolymerization of a Supported Lipid Film.

Details of the photopolymerization of lipid films on alkylated glass and silicon have been reported elsewhere. (Orban, J. M. et al., Macromolecules 2000, 33, 4205-4212; Liu, H. et al., Langmuir 2002, 18:1332-1339.) Briefly, a stock solution of coinitiators was prepared as 10 mM Eosin Y (EY), 225 mM triethanolamine (TEA), and 37 mM VP in water. A 10:1 (mol/mol) monomer/EY ratio was used for photopolymerization. After lipid fusion, the samples were placed into a N₂-purged atmosphere at 30% relative humidity and 10 μL of initiator was added per 1 mL of sample solution. The initiator was gently mixed by slowly rotating the vial in a horizontal circular motion without lifting it from the bench surface. The sample was then irradiated with a Dynalume visible light lamp at an intensity of 50 mW/cm². Following photopolymerization, the samples were washed with deionized water 6-8 times and stored for analysis.

Generation of a Glycocalyx-Mimetic Surface on a Supported Lipid Membrane:

Streptavidin Binding onto a Biotin-Functionalized Lipid Membrane.

Streptavidin and FITC-labeled streptavidin, for confocal fluorescence microscopy studies, were prepared in PBST (150 mM NaCl, 50 mM NaH₂PO₄, pH 7.34) at a concentration of 5 μL. Streptavidin-containing solutions were incubated with biotin-functionalized substrates for 15 min with horizontal shaking, followed by rinsing 10 times with deionized water.

Glycopolymer Binding to a Streptavidin-Functionalized Surface.

Streptavidin-coated lipid membranes were incubated with biotin-terminated glycopolymer (25 μg/mL) in PBST for 1 h at room temperature with horizontal shaking. The membrane was then washed 10 times with deionized water. For confocal fluorescence microscopy studies, the film was incubated with FITC-labeled P. tetragonolobus lectin (100 μg/mL) in PBST for 1 h at room temperature with horizontal shaking and then washed 10 times with deionized water.

A chain-end-functionalized glycopolymer was synthesized in which multivalent lactose units serve as ligands for lectins and/or antibodies and a single biotin group on the initiating species provides a convenient anchor to avidin or streptavidin. Modulating lactose density and polymer solubility was achieved by using acrylamide as a comonomer. Binding specificity to streptavidin has been previously verified using a SDS-PAGE gel shift assay and confocal fluorescence imaging of the glycopolymer adsorbed onto a streptavidin-pattemed surface (see Example 1).

Fabrication of a Glycocalyx-Mimetic Surface using Biotin-Streptavidin Coupling to a Supported Lipid Film:

We have demonstrated in prior reports that membrane-mimetic films that are stable in air and under static and dynamic flow conditions in an aqueous environment can be produced by in situ polymerization of a planar lipid assembly on a variety of alkylated supports. (Liu, H. et al., Langmuir 2002, 18, 1332-1339.) Significantly, an alkylated terpolymer, electrostatically coupled to a polyelectrolyte multilayer, allows film fabrication on a hydrophilic cushion that facilitates the incorporation of transmembrane proteins. In this investigation, mixed vesicles, comprised of polymerizable lipids with either biotin or phosphatidylcholine headgroups, were fused onto an alkylated terpolymer bound to a poly-L-lysine/alginate multilayer. Streptavidin and subsequent glycopolymer coating of the polymerized lipid film was then performed to produce a glycocalyx mimic.

Contact angles were measured at various stages for films containing 10, 25, or 50 mol % biotin-PE/PC lipids (B-PE/PC) (FIG. 2). In agreement with prior studies, advancing contact angles for the ALG/PLL multilayer were low (35°) and increased after the addition of the terpolymer (105-109°). Contact angles for films composed solely of lipids containing a phosphatidylcholine headgroup characteristically range between 50 and 60°. (Orban, J. M. et al., Macromolecules 2000, 33, 4205-4212.) The addition of biotinylated lipids, however, produced a small but noticeable increase in film hydrophobicity, with contact angles of 51, 59, and 77° for films containing 10, 25, and 50 mol % of biotin-PE, respectively. Advancing contact angles were unchanged upon addition of streptavidin or glycopolymer to 10 or 25 mol % biotin-PE/PC films. A reduction in film hydrophobicity from 77° to 65° was observed after incubation of the 50 mol % biotin-PE/PC sample with streptavidin. Glycopolymer coating, even at high surface binding density, was not associated with further reduction in contact angle.

Structural Characterization of a GlycocalyxMembrane-Mimetic Film Using Polarized Infrared External Reflection Spectroscopy:

The Effect of Increasing Biotin-PE Film Concentration.

Polarized external reflection infrared spectra were acquired during each stage of film construction in order to identify functional group characteristics and induced structural changes unique to each film component. Specifically, infrared spectroscopy provides detailed information regarding hydrocarbon chain conformation and order and, by using polarized spectra, molecular orientation. The level of disorder may influence film properties, such as diffusion across a lipid membrane, as well as the orientation and assembly of membrane-associated proteins. Characteristic IR absorption bands of the component structures of the fabricated glycocalyx-mimetic film are summarized in Table 1.

TABLE 1
Infrared Band Assignments for Components Present
in a Glycocalyx-Mimetic Thin Film

| absorption mode | frequency (cm⁻¹) | component of film |
|---|---|---|
| OH stretch | 3600-3000 | ALG, GLY |
| amide A (mostly N-H) | ˜3300 | PLL, STREP |
| amide B | ˜3100 | STREP |
| CH₂ stretch (antisymm) | 2926-2918 | TER, B-PE, PC |
| CH₂ stretch (symm) | 2853-2850 | TER, B-PE, PC |
| C═O (ester) | 1735 | TER, B-PE, PC |
| amide I | 1650 | PLL, STREP |
| COO— stretch (antisymm) | 1602 | ALG |
| amide II | 1550 | PLL, STREP |
| CH₂ bend (scissoring) | 1456 | TER, B-PE, PC |
| COO— stretch (symm) | 1402 | ALG |
| PO₂— stretch (antisymm) | ˜1263-1245 | B-PE, PC (stronger) |
| C—O—C stretch | ˜1225-1217 | TER, B-PE, PC, GLY |
| (antisymm) | | |
| C—O—O—C stretch | ˜1171 | TER, B-PE, PC |
| (antisymm) | | |
| C═O—O—C stretch ˜ | 1100 | TER, B-PE, PC |
| (symm) | | |
| PO₂— stretch (symm) | ˜1090 | B-PE, PC (stronger) |
| C—O—C stretch (symm) | ˜1040-1030 | TER, B-PE, PC, GLY |
| (CH₃)₃N⁺ bend (antisymm) | 975 | PC |
| (CH₃)₃N⁺ bend (symm) | 925 | PC |
| P—O stretch (symm) | 816 | B-PE, PC |

Abbreviations:
antisymm, antisymmetric;
symm, symmetric;
PLL, poly-L-lysine;
ALG, alginate;
TER, poly(HEA₆-AOD₃-SSS₁) terpolymer;
B-PE, biotin-mono-acrylPE;
PC, mono-acrylPC;
STREP, streptavidin;
GLY, glycopolymer.

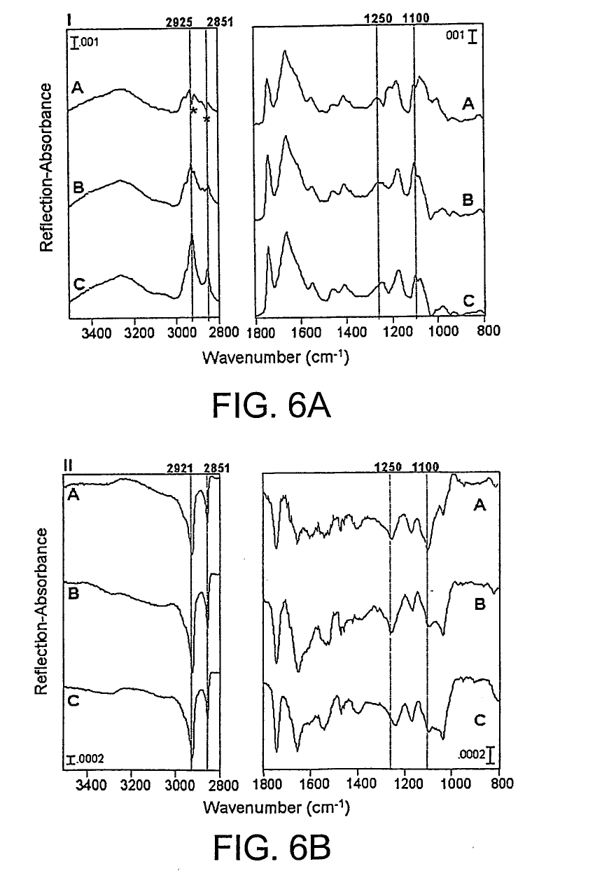

Varying the surface concentration of biotin-PE yields several changes in Rs and Rp spectra (FIG. 6). On increasing biotin-PE content from 10 to 25 mol %, a decrease is observed in the combination of symmetric C═O—O—C and PO₂— stretches at ˜1100 cm⁻¹ in the Rs polarized spectrum (FIG. 6II, A vs B). In addition, increasing biotin-PE composition is associated with a broadening of the antisymmetric PO₂— headgroup band at 1250 cm⁻¹ that appears to shift to a lower wavenumber in both Rp and Rs polarized spectra (FIG. 6I, II, A-C). Reference ATR-IR spectra of acrylate-PC and biotinacrylate-PE in powder form demonstrate that both compounds possess PO₂— antisymmetric (˜1245 cm-1) and symmetric (1090 cm⁻¹) stretches, although the intensities of these bands are significantly reduced in biotin-PE. Thus, it is likely that the observed reduction in intensity at 1100 cm⁻¹ in the Rs polarized spectra results from a direct change in film composition (i.e., a reduction in acrylate-PC). Likewise, the shift and broadening of the band at ˜1250 cm⁻¹ to a lower wavenumber is due to both a reduction of the PO₂— antisymmetric stretch and a simultaneous increase of the C—O—C stretch at 1217 cm⁻¹.

The most notable difference in the polarized IR spectra on varying biotin concentration occurs in the va and vs CH₂ stretching modes from 3000 to 2800 cm⁻¹ of the Rp polarized spectra, which shift from negative to positive with increasing biotin-PE concentration (FIG. 6I, A-C). The observation of positive and negative absorption bands in polarized spectra has been previously reported in theoretical and experimental studies of monomolecular thin films coated onto silicon substrates. We have also observed this behavior in polymeric lipid films deposited onto OTS/Si and onto (PLL/ALG)5-PLL-poly-(HEA6-DOD3-SSS1) terpolymer/Si coated substrates. Using the Fresnel reflection equations for a three-phase system (Dluhy, R. A., J. Phys. Chem. 1986, 90, 1373-1379; Mielczarski, J. A. and Yoon, R. H., J. Phys. Chem. 1989, 93, 2034-2038), we correlated, qualitatively and quantitatively, the positive and negative methylene absorption modes with the molecular orientation of the alkyl chains in the OTS, terpolymer, and supported lipid monolayers. (Orban, J. M. et al., Macromolecules 2000, 33, 4205-4212; Liu, H. et al., Langmuir 2002, 18, 1332-1339.) From these investigations, we correlated the positive intensity of the Rp polarized va and vs CH₂ stretching modes with a random alkyl chain orientation that is tilted away from the surface normal and negative CH₂ stretching modes with more ordered alkyl chain packing. In this regard, the negative CH₂ absorption bands observed in the 10 mol % B-PE/PC film (FIG. 6I, A) are indicative of ordered alkyl chains. An ordered alkyl chain orientation is also supported by the frequency positions of the methylene stretching modes, which can be utilized to assess hydrocarbon chain order. The va and vs CH₂ positions of 2919 and 2851 cm⁻¹, respectively, indicate that the alkyl chains are only slightly disordered and possess a predominance of trans conformers.

Increasing the concentration of biotin-PE induces alkyl chain disorder. Specifically, in films containing 25 mol % of biotin-PE, the intensity of methylene absorption modes shifts from negative to positive, indicating an increase in random chain orientation (FIG. 6I, B). In support of this notion, the va CH₂ band also increases from 2919 to 2920 cm⁻¹. Further, in films containing 50 mol % of biotin-PE (FIG. 6I, C), methylene bands are positive and the va CH₂ band shifts to 2925 cm⁻¹, consistent with disordered hydrocarbon chains containing large numbers of gauche conformers.

Binding of Streptavidin and Glycopolymer to Biotin-Functionalized Surfaces.

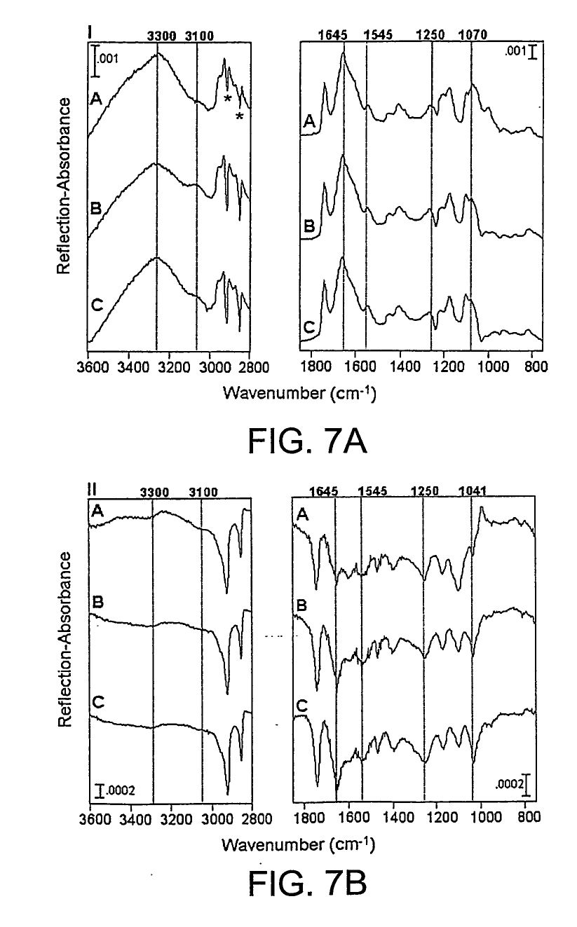

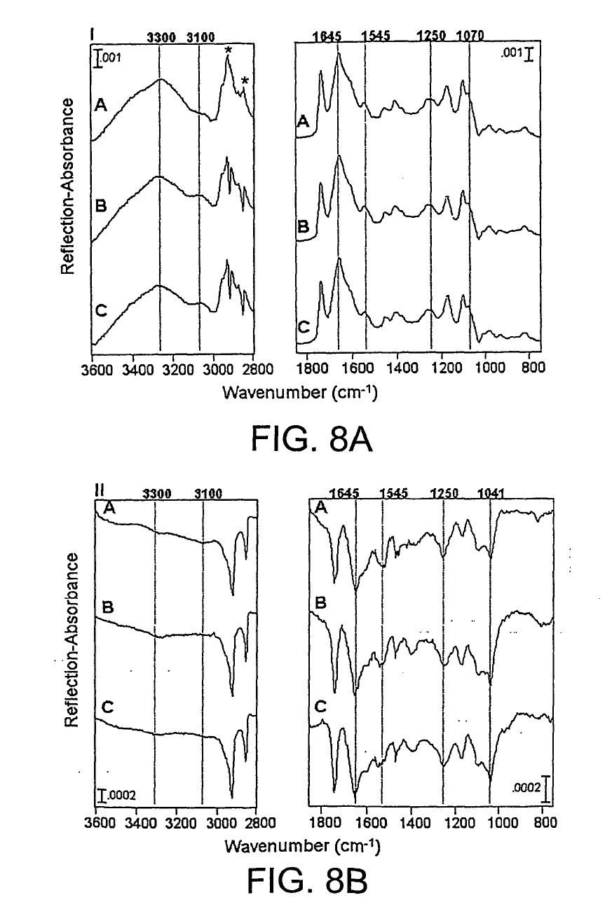

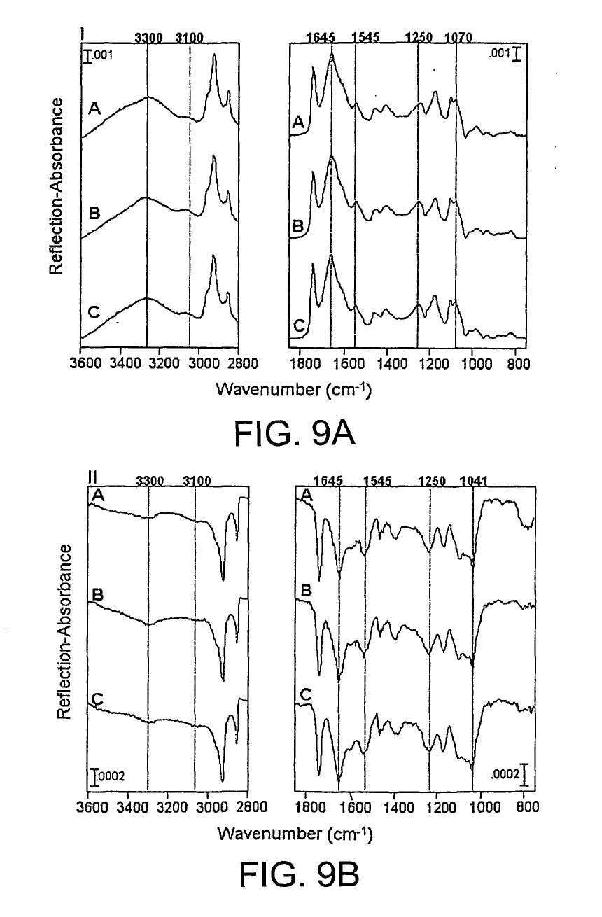

Polarized IR spectra obtained after streptavidin binding are presented in FIGS. 7-9, spectra B. The presence of streptavidin is represented by the appearance of amide A (˜3300 cm-1) and amide B (˜3100 cm⁻¹) bands. An additional observation is the increase in the negative intensity of the Rp polarized methylene absorption bands for both the 10 and 25 mol % B-PE/PC films that represent induced alkyl chain orientation. No appreciable changes were noted in IR spectra after glycopolymer binding (FIGS. 7-9, spectra C).

Fluorescence Imaging of Streptavidin and Glycopolymer Bound onto Membrane-Mimetic Films:

Confocal fluorescence images obtained after incubation of biotin-derivatized films with FITC-labeled showed, as expected, that surface fluorescence increases with increasing concentrations of biotin-PE. Of interest, microdomains of FITC-labeled streptavidin appear on films containing 50 mol % biotin-PE and are thought to indicate a clustering of biotin-PE. The inhomogeneity of the observed fluorescence is related to microphase separation of the PC and biotinylated lipids. Increasing alkyl chain disorder along with a small but noticeable increase in film hydrophobicity is indicative of incomplete vesicle fusion at high biotin-PE surface concentrations.

Fluorescence images after incubation with FITC-labeled lectin show that glycopolymer-modified surfaces demonstrate a much greater degree of homogeneous fluorescence at 25 and 50 mol % biotin-PE labeling when compared to the corresponding streptavidin-decorated surfaces. This can be attributed to the large amplification of lectin binding sites over the film surface due to the presence of several biotin binding sites on each streptavidin molecule, as well as the availability of approximately 10 pendent lactose units per glycopolymer chain. Relatively little nonspecific lectin or streptavidin binding was noted on membranes composed of phosphatidylcholine lipids alone.

### EXAMPLE 3

Glycopolymers of Sulfated Lactose Exhibit Anticoagulant Activity.

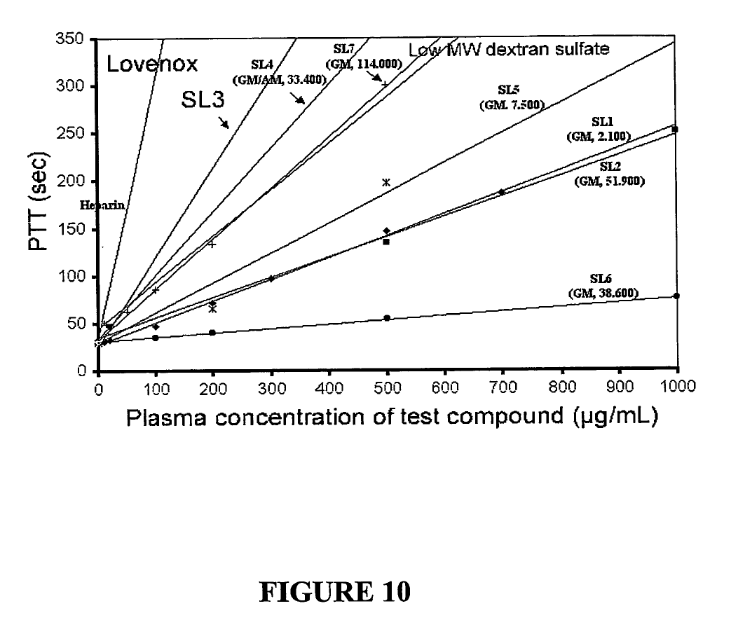

Glycopolymers of varying molecular weight have been synthesized, as described above, bearing varying backbone densities of sulfated lactose pendent groups. These compounds have been fully characterized by NMR and mass spectroscopy and anticoagulant properties defined using an activated partial thromboplastin time (APTT) assay. Results demonstrate that anticoagulant activity is structure dependent and approaches activity levels observed for heparin and heparin-related compounds. FIG. 10 shows results using a glycopolymer designated SL3 (MW 9300, PDI 1.46) which was a copolymer of sulfated lactose monomers (GM) and acrylamide (AM) (molarratio AM:GM 1:10, lactose content 57 wt %). Compare with Lovenox control).

### EXAMPLE 4

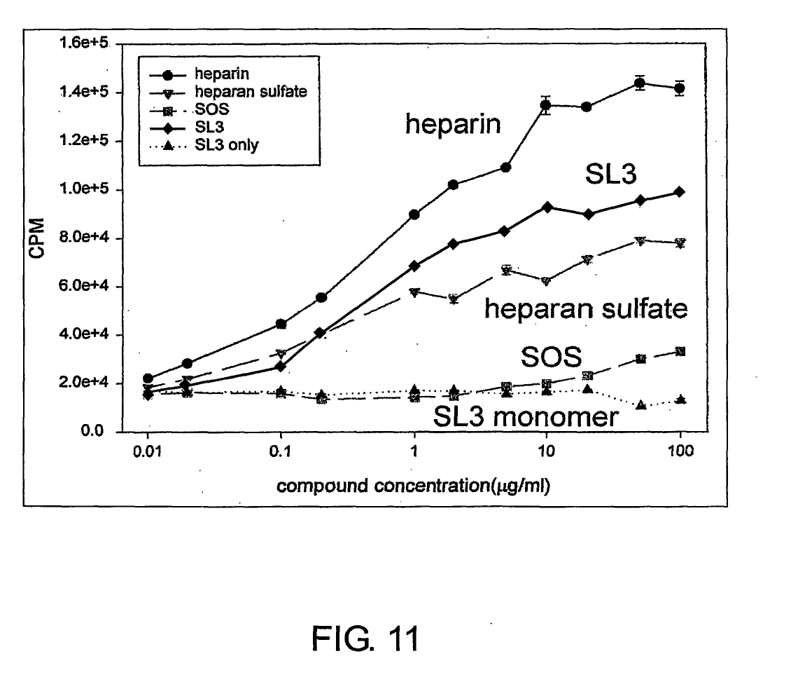

Glycopolymers bearing lactose heptasulfate dissacharides can stimulate cell proliferation and mediate dimerization of growth factor FGF-2 and growth factor receptor FGFR-1. The capacity of selected glycopolymers to potentiate FGF-2 mitogenic activity was measured by [³H]thymidine incorporation into BaF3-FR1 C-11 cells, which express the FGF receptor, FGFR-1, but not cell surface heparan sulfate. Cells were treated with 3 ng/mL of FGF-2 and increasing concentrations of indicated oligosacharides. Test compounds include a series of glycopolymers ranging in molecular weight, pendent group type and density, as well as corresponding monomers, sucrose octasulfate (SOS), heparin (Sigma porcine intestinal MW 12,000-20,000), and heparan sulfate (Sigma MW 7,000). Glycopolymers carrying fully sulfated NAcGlc pendent groups, for example, were ineffective in promoting FGF-2 dependent cell proliferation. However, glycopolymers bearing sulfated lactose residues were able to elicit a range of proliferative activity depending upon the structural features of the polymer (FIG. 11). The highest level of bioactivity was demonstrated by SL3-mediated proliferative responses to FGF-2 exceeded those observed with heparan sulfate, as well as SOS and free SL3 monomer. Of interest, the SL3 effect is selective in that it was not capable of initiating a proliferative response to FGF-1.

### EXAMPLE 5

In order to examine the capacity of SL3 to mediate FGF-2 dimerization, 20 ng/mL of ¹²⁵I-FGF-2 was incubated with either heparin or SL3 for 1 h at room temperature. After disuccinimidyl suberate cross-linking, the samples were resolved by electrophoresis and visualized by autoradiography. SL3 was as effective as heparin in mediating FGF-2 dimerization. Likewise, SL3 mediated FGFR-1 dimerization was investigated by incubating BaF3-FGFR-1C-11 cells with ¹²⁵I-labeled FGF-2 in the presence or absence of either heparin or SL3. Cells were treated with disuccinimidyl suberate to cross-link FGF-2 to its receptor. Crosslinked proteins were electrophoresed on an SDS-6% polyacrylamide gel and detected by autoradiography. SL3 was as effective as heparin in mediating FGFR-1 dimerization. Both FGF-2 and FGFR-1 dimerization are essential for an optimized biological response. It is also noteworthy that FGF-2 is characteristically sequestered in the ECM in a dimerized form.

### EXAMPLE 6

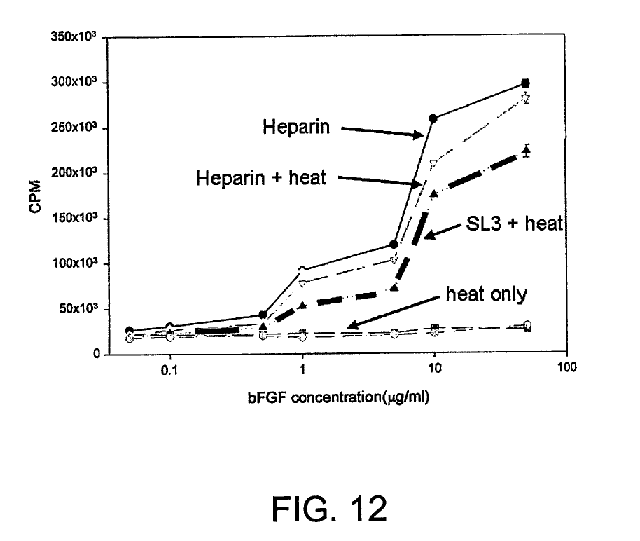

Sulfated lactose glycopolymers protect FGF-2 from protein degradation induced by trypsin, acidic conditions, and heat. It has been suggested that once soluble proteins, such as FGF-2, are released into the extra-cellular matrix, their sequestration by heparan sulfate proteoglycans is essential for preservation of bioactivity. In order to assess protection from proteolysis, FGF-2 (1 μg) was incubated with trypsin for 3 h at 37° C. in the presence or absence of 25 μg/mL of heparin or decreasing concentration of glycopolymer SL3. Samples were then analyzed by SDS-PAGE and undigested FGF-2 was estimated by densitometry. SL3 was as effective as heparin in protecting FGF-2 from proteolytic degradation. Protection from acidic conditions was determined by incubation of FGF-2 with or without heparin or glycopolymer (25 μg/mL) in varying concentrations of trifluoroacetic acid (0.05 to 5%), corresponding to a pH range of 0 to 3.4 (133). After a two hour incubation at room temperature, samples were diluted 20 fold with RPMI 1640 and incubated with heparin deficient cells (BaF3-FGFR-1) expressing the FGF receptor (FGFR-1). ³H-thymidine incorporation was determined as an indicator of retained FGF-2 activity. Preservation of biological activity upon heat-treatment of FGF-2 was measured by incubation of FGF-2 at 65° C. for 5 min with or without heparin or glycopolymer (25 μg/mL). Samples were diluted in RPMI 1640 and incubated with BaF3-FGFR-1 cells and ³H-thymidine incorporation determined (FIG. 12). Both heparin and the heparin-mimetic glycopolymer provided substantial protection of FGF-2 from trypsin, heat, and acidic conditions.

Although this invention has been illustrated with specific molecules, reagents and reaction conditions, it will be appreciated by those of skill in the art that other such molecules, reagents and reaction conditions may be used or generated, and such equivalents are considered to be within the scope of the following claims.

1. A molecule comprising:
a polymer backbone;
pendent multivalent groups attached to said polymer backbone; and
an anchoring group attached to said polymer backbone for covalently or noncovalently attaching the molecule to a surface or another molecule.

2. The molecule of claim 1 wherein said pendent multivalent group is a saccharide.

3. The molecule of claim 2 wherein said saccharide is selected from the group consisting of monosaccharides, disaccharides) trisaccharides and oligosaccharides, and such saccharides in which one or more OH moieties are replaced by SO₃.

4. The molecule of claim 2 wherein said saccharide is selected from the group consisting of N-acetyl-D-glucosamine, α- and β-N-acetyl-D-glucosamine-(1-4)-β-D-glucuronic acid, pyranosides, lactose, and polylactose.

5. The molecule of claim 1 wherein said pendent multivalent group is selected from the group consisting of glycosaminoglycans, sialic acid, siayl Liews X, heparin, and oligopeptides.

6. The molecule of claim 1 which has a polydispersity index (molecular weight Mw/molecular number Mn) between about 1.1 and about 1.5.

7. The molecule of claim 1 comprising between about 2 and about 1000 pendent multivalent groups.

8. The molecule of claim 1 also comprising a spacer arm between said anchoring group and said polymer backbone.

9. The molecule of claim 8 wherein said spacer arm comprises a ring.

10. The molecule of claim 8 wherein said spacer arm comprises a phenyl ring.

11. The molecule of claim 1 wherein said spacer arm comprises a group selected from the group consisting of arylamide, methacrylamide, acryloyl, methacrylol, and vinyl.

12. The molecule of claim 1 wherein said anchoring group is selected from the group consisting of biotin, biotin-cap, charged anchoring groups, chemically-reactive groups, photocrosslinkable groups, and hydrophobic groups.

13. The molecule of claim 12 wherein said charged anchoring group is selected from the group consisting of styrene sulfate, styrene sulfonate, alkyl sulfate, alkyl sulfonate, and alkyl sulfonic acid.

14. The molecule of claim 12 wherein said chemically-reactive group is selected from the group consisting of a thiol, an amine, an aldehyde and a carboxylic acid.

15. the molecule of claim 12 wherein said photocrosslinkable group is selected from the group consisting of aryldiazirine and arylazide.

16. The molecule of claim 12 wherein said hydrophobic group is selected from the group consisting of C₆-C₂₂ alkyl, C₁₂-C₂₂ lipid and C₁₂-C₂₂ phospholipid.

17. The molecule of claim 1 comprising a first pendent unit comprising a linking group connected to said polymer backbone and a multivalent moiety connected to said linking group.

18. The molecule of claim 17 also comprising a second pendent unit comprising a lipid or long alkyl chain of about C₇ to C₃₀.

19. The molecule of claim 17 wherein the number of pendent units is up to about 2000.

20. The molecule of claim 18 wherein said second pendent unit comprises phosphatidylcholine.

21. The molecule of claim 1 also comprising a cyanoxyl group at the end of the polymer backbone opposite said anchoring group.

22. A bioactive surface comprising the molecule of claim 1 covalently or non-covalently bound to said surface via said anchoring group.

23. The surface of claim 22 selected from the group consisting of metal, glass, silicon, synthetic polymers, natural polymers, surfaces of medical devices for contact with blood or tissue, and membrane mimetic surfaces.

24. The surface of claim 23 wherein said natural polymers are selected from the group consisting of alginates and collagen.

25. The surface of claim 23 wherein said medical devices are selected from the group consisting of vascular grafts, catheters, and biosensors.

26. The surface of claim 23 which is a membrane mimetic surface selected from the group consisting of glycocalyx-mimetic surfaces, supported lipid surfaces, and polyelectrolyte multilayers

27. The surface of claim 23 which is an elastic polypeptide block copolymer having hydrophilic and hydrophobic groups.

28. The molecule of claim 1 wherein said anchoring group is covalently bound to a binding partner.

29. The molecule of claim 28 wherein said binding partner is selected from the group consisting of: avidin, streptavidin, and an amine-containing group which links via —OCN by isourea bond formation.

30. The molecule of claim 28 wherein said amine-containing group is selected from the group consisting of a protein, a polypeptide, a polymer, a dendrimer, and a lipid.

31. A molecule comprising 2 to 4 molecules of claim 1 covalently bound to a binding partner.

32. A molecule of claim 1 covalently or non-covalently bound to a biological marker.

33. The molecule of claim 32 wherein said marker is an organic or inorganic fluorescent dye or nanoparticle.

34. A molecule of claim 1 having a molecular weight of about 9300 and a PDI of 1.46 which is a copolymer of sulfated lactose monomers and acrylamide at a molar ratio of acrylamide to sulfated lactose monomers of about 1:10 and a lactose content of about 57 wt %.

35. A method for reducing coagulation of blood comprising contacting said blood with an effective amount of a molecule of claim 1 which is a sulfated glycopolymer.

36. The method of claim 35 wherein said molecule is a molecule having a molecular weight of about 9300 and a PDI of 1.46 which is a copolymer of sulfated lactose monomers and acrylamide at a molar ratio of acrylamide to sulfated lactose monomers of about 1:10 and a lactose content of about 57 wt %.

37. A method of selectively stimulating FGF-2-dependent cell proliferation comprising contacting cells with an effective amount of a sulfated molecule of claim 1.

38. The method of claim 37 wherein said sulfated molecule is a heptasulfate disaccharide.

39. The method of claim 37 wherein said sulfated molecule is a molecule having a molecular weight of about 9300 and a PDI of 1.46 which is a copolymer of sulfated lactose monomers and acrylamide at a molar ratio of acrylamide to sulfated lactose monomers of about 1:10 and a lactose content of about 57 wt %.

40. A method of preventing degradation of soluble growth factors released into extra-cellular matrix comprising contacting said growth factors with an effective amount of a molecule of claim 1 comprising a sulfated saccharide.

41. The method of claim 40 wherein said molecule is is a molecule having a molecular weight of about 9300 and a PDI of 1.46 which is a copolymer of sulfated lactose monomers and acrylamide at a molar ratio of acrylamide to sulfated lactose monomers of about 1:10 and a lactose content of about 57 wt %.

42. A method of making a molecule of claim 1 comprising the steps of:
(a) providing a compound having the structure: 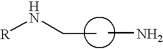
wherein R is an anchoring group; and
◯ represents a ring selected from the group consisting of aryl, aralkyl, cycloalkyl, cycloalkenyl, heterocyclic rings and substituted rings;
(b) reacting said molecule of step (a) with HBF₄ to form a molecule having the structure: 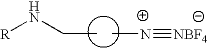
(c) reacting the molecule of step (b) with a cyanate to form a molecule having the structure: 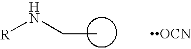
wherein R is an anchoring group, and
(d) reacting the molecule of step (c) with a molecule having a multivalent moiety and a terminal vinyl group to form a compound having the formula: 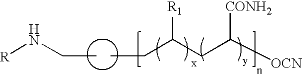
wherein x is between about 1 and about 50, preferably between about 5 and about 30, and more preferably between about 5 and about 12;
y is between about 1 and about 100, preferably between about 50 and about 80;
n is between about 1 and about 100, preferably between about 10 and 50;
R₁ is selected from the group consisting of monosaccharides, oligosaccharides, polysaccharides and glycosaminoglycans such as galactose, sialic acid, siayl Liews X, heparin, oligopeptides and other biomolecules, conjugated to —(CH₂)m-HN—CO— which is conjugated to the polymer backbone; and
wherein m is 1 to about 5.

43. The method of claim 42 wherein a comonomer comprising a vinyl group is added to the reaction of step (d).

44. The method of claim 43 wherein said comonomer is selected from the group consisting of acrylamide, acrylate, and C₁-C₁₀ alkenyl.

45. A molecule having the following formula: 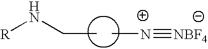
wherein R is an anchoring group, and ◯ represents a ring selected from the group consisting of aryl, aralkyl, cycloalkyl, cycloalkenyl, heterocyclic rings and substituted rings.

46. A molecule having the following formula: 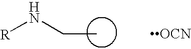
wherein R is an anchoring group, and ◯ represents a ring selected from the group consisting of aryl, aralkyl, cycloalkyl, cycloalkenyl, heterocyclic rings and substituted rings.

47. A molecule having the following formula: 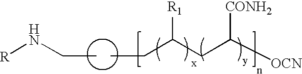
wherein R is an anchoring group;
◯ represents a ring selected from the group consisting of aryl, aralkyl, cycloalkyl, cycloalkenyl, heterocyclic rings and substituted rings;
x is between about 1 and about 50, preferably between about 5 and about 30 and more preferably between about 5 and about 12;
y is between about 1 and about 100, preferably between about 50 and about 80;
n is between about 1 and about 100, preferably between about 10 and 50;
and R₁ is selected from the group consisting of monosaccharides, oligosaccharides, polysaccharides and glycosaminoglycans such as galactose, sialic acid, siayl Liews X, heparin, oligopeptides and other biomolecules, conjugated to —(CH₂)m-HN—CO— which is conjugated to the polymer backbone; and
wherein m is 1 to about 5.
